(19) United States
(12) Patent Application Publication
(10) Pub. No.: US 2009/0299364 A1
(43) Pub. Date: Dec. 3, 2009

(54) Suction Force Ablation Device

(75) Inventors: Kester Batchelor, Minneapolis, MN (US); David Francischelli, Anoka, MN (US); Tom Daigle, Hanover, MN (US); Dan Haeg, Champlin, MN (US); Paul Rothstein, ELK River, MN (US); Adam Podbelski, St. Paul, MN (US); Sarah Ahlberg, Crystal, MN (US); Steve Ramberg, North Oaks, MN (US); Tom Conway, White Bear Lake, MN (US)

Correspondence Address: Medtronic CardioVascular, Mounds View Facility South, 8200 Coral Sea Street N.E., Mounds View, MN, 55112, US

(73) Assignee: Medtronic, Inc., Minneapolis, MN (US)

(21) Appl. No.: 12/427,457

(22) Filed: Apr. 21, 2009

Related U.S. Application Data
(60) Provisional application No. 61/046,531, filed on Apr. 21, 2008.

(51) Int. Cl. A61B 18/14

(52) U.S. Cl. 606/41

(57) ABSTRACT

An ablation device for ablating tissue having an outer wall and an inner wall, approximately parallel and concentric with said outer wall, defining an inner fluid chamber and an outer low pressure chamber. Each of the outer wall and the inner wall have an edge defining an open face of the fluid chamber and the low pressure chamber. An ablative element is contained within the fluid chamber. A source of low pressure is coupled to the low pressure chamber. When the edge of the outer wall and the edge of the inner wall contact a surface, the ablation device is at least partially secured to the surface by low pressure created in the low pressure chamber by the source of low pressure. The fluid chamber is at least partially fluidly isolated from the low pressure chamber when the ablation device is at least partially secured to the surface.

### RELATED APPLICATION

This application claims the benefit under 35 U.S.C. §119(e) of U.S. Provisional Patent Application No. 61/046,531, filed Apr. 21, 2008, which is incorporated herein by reference in its entirety.

### FIELD

This disclosure relates to ablation devices that are used to create lesions in tissue. More particularly, this disclosure relates to ablation devices that use a suction force to hold the device against the tissue while the device creates lesions.

### BACKGROUND

The action of the heart is known to depend on electrical signals within the heart tissue. Occasionally, these electrical signals do not function properly. Ablation of cardiac conduction pathways in the region of tissue where the signals are malfunctioning has been found to eliminate such faulty signals. Ablation is also used therapeutically with other organ tissue, such as the liver, prostate and uterus. Ablation may also be used in treatment of disorders such as tumors, cancers or undesirable growth.

Sometimes ablation is necessary only at discrete positions along the tissue, is the case, for example, when ablating accessory pathways, such as in Wolff-Parkinson-White syndrome or AV nodal reentrant tachycardias. At other times, however, ablation is desired along a line, called a linear ablation. This is the case for atrial fibrillation (AF), where the aim is to reduce the total mass of electrically connected atrial tissue below a threshold believed to be critical for sustaining multiple reentry wavelets. Linear transmural lesions are created between electrically non-conductive anatomic landmarks to reduce the contiguous atrial mass. Transmurality is achieved when the full thickness of the target tissue is ablated.

Ablation is performed during surgery, and is currently accomplished in several ways. One way is to position a tip portion of the ablation device so that an ablation electrode is located at one end of the target site. Then energy is applied to the electrode to ablate the tissue adjacent to the electrode. The tip portion of the electrode is then slid along the tissue to a new position and then the ablation process is repeated. A second way of accomplishing linear ablation is to use an ablation device having a series of spaced-apart band or coil electrodes that, after the electrode portion of the ablation device has been properly positioned, are energized simultaneously or one at a time to create the desired lesion. If the electrodes are close enough together, the lesions run together sufficiently to create a continuous linear lesion.

Recent advances in surgical techniques have been directed to minimally invasive procedures that can reduce patient discomfort, reduce recovery time, and often reduce complexity. In the case of cardiac surgery, minimally invasive procedures are often favored over open surgical procedures, and procedures on a beating heart are often favored over procedures on an arrested or stopped heart. In these cases ablation devices can be directed to work with minimally invasive introducer devices and directed to secure themselves to a beating heart.

Although these types of ablation devices are known, they may be unable to effectively secure the electrode to a beating heart, or the securing mechanism may errantly affect the electrode. In a common example, the electrode is saline from a saline eluting polymer, where the saline is energized to create a lesion near the target site. Also in a common example, the securing mechanism is a suction port connected to the electrode to hold the electrode in place against the beating heart.

Several potentially undesirable effects are possible with this configuration and are observed in prior art examples. For example, if the saline were to be in fluid contact with the suction, the low pressure of the suction may lower the boiling point of the saline. At atmospheric pressure near sea level, saline has a boiling point at just above the boiling point of water, such as 102 degrees Celsius. Typical United States operating rooms include a suction source at a relative pressure of 300 mm/Hg, which is connected to the suction port on the ablation device. At this relative pressure, the boiling point of saline can drop to 85 degrees Fahrenheit, or 17 degrees Celsius.

Lowering the boiling point of saline may cause a faster phase change in the saline, which may occur well before ablation can be successful. The phase change may cause micro bubbles, steam, and air in the tissue to separate from the tissue, which may cause to separate the ablation device from the tissue. The suction on the tissue may also cause the lowering of the boiling point on the tissue, which has been demonstrated to damage the tissue in an unintended manner from the lesions, which can be counteractive to eliminating faulty electrical signals of the heart.

Other example ablation devices include disposing the suction port and electrode in separate chambers. In one example, suction ports are positioned generally proximate to the electrode, which is positioned on top of the patient tissue. These devices can suffer from an imbalance of pressures on the device between the suction and the volume of saline delivery coupled with the variable of power delivery. The devices can be subject to high impedance shut off (HISO) due to the imbalance. For example, HISO is created when the electrode is held too tightly against the tissue and the saline boils to create micro bubbles of relatively high electrical resistivity. When power is reduced, transmurality of the tissue may not be achieved within an optimal amount of time from activation. Also, the devices can be structurally unstable due to the imbalance, which may lead to twisting and liftoff again contributing to HISO conditions. These examples of devices separating the suction port from the electrode may have unpredictable ablation results.

### SUMMARY

An ablation device has been created which has a low pressure chamber arrayed concentrically around a fluid chamber, which includes an ablation element, into which fluid may be introduced to aid the ablation process. By creating a concentric low pressure chamber, the ablation device may be better secured to patient tissue than may be possible with conventional ablation devices. By providing a securing force all the way around the fluid chamber, the electrode may be maintained a more uniform distance from the tissue than may otherwise be created, and may be more resilient against forces which may tend to cause the ablation device to separate from the patient tissue at an unwanted time.

In an embodiment, a suction force ablation device suitable for use with an organic tissue has a first recessed area configured to form a first chamber when the device is urged against the tissue and a second recessed surface configured to form a second chamber, concentric with the first chamber, such that when the device is urged against the tissue the first and second chambers are fluidically isolated from each other. The device also has an electrode disposed within the second chamber, and the first chamber is under a low pressure than the second chamber and is configured to provide a suction force against the tissue.

In an embodiment, the first recessed area and the second recessed areas are each surrounded by a wall.

In an embodiment, the wall is configured to be flexible and conform to the tissue.

In an embodiment, the wall is constructed from a flexible closed cell foam.

In an embodiment, a common wall separated the first recessed area and the second recessed area.

In an embodiment, the first recessed area surrounds the second recessed area.

In an embodiment, the electrode is electrically coupled to a power source.

In an embodiment, an irrigation fluid is introduced into the first chamber from an irrigation source.

In an embodiment, the electrode is a fluid eluting polymer fluidly coupled to the irrigation source.

In an embodiment, a suction force ablation device suitable for use with an organic tissue has a housing having first upstanding wall and a second upstanding wall, the first upstanding wall being concentric with the second upstanding wall, a first recessed portion surrounded by the first wall, an electrode disposed within the first wall, and a vent within the first wall, and the vent is in fluid communication with atmospheric pressure through another vent on the housing. The device also has a second recessed portion surrounded by the first wall and the second wall, and a suction port disposed between the first and second walls, and the suction port is configured to provide a suction force.

In an embodiment, the second wall surrounds the second recessed layer and the first wall.

In an embodiment, the device is configured to be urged against tissue and form a first chamber surrounded by the first wall and the second wall and a second chamber surrounded by the first walls, and the first chamber surrounds the entire second chamber.

In an embodiment, the housing further includes a connector portion.

In an embodiment, an ablation device for ablating tissue has an outer wall and an inner wall, approximately parallel and concentric with the outer wall, defining an inner fluid chamber and an outer low pressure chamber, each of the outer wall and the inner wall having an edge defining an open face of the fluid chamber and the low pressure chamber. The ablation device also has an ablative element contained within the fluid chamber, and a source of low pressure coupled to the low pressure chamber. When the edge of the outer wall and the edge of the inner wall contact a surface, the ablation device is at least partially secured to the surface by low pressure created in the low pressure chamber by the source of low pressure. The fluid chamber is at least partially fluidly isolated from the low pressure chamber when the ablation device is at least partially secured to the surface.

In an embodiment, the ablation device also has a source of fluid delivered to the fluid chamber.

In an embodiment, the source of fluid comprises a reservoir fluidly coupled to the fluid chamber.

In an embodiment, the fluid is a conductive fluid.

In an embodiment, the ablation device also has a fluid removal lumen fluidly coupled to the fluid chamber.

In an embodiment, a fluid chamber pressure is greater than a low pressure chamber pressure.

In an embodiment, the outer wall comprises a flange which contacts the surface.

In an embodiment, the flange is flexible.

In an embodiment, the flange is curved.

In an embodiment, the outer wall has a bellows.

In an embodiment, the bellows is a single bellows.

In an embodiment, the bellows is a double bellows.

In an embodiment, the low pressure chamber is defined by a gap between the outer wall and the inner wall and the fluid chamber is defined by the inner wall.

In an embodiment, the low pressure chamber forms a concentric ring around the fluid chamber.

In an embodiment, the ablative element comprises an electrode.

In an embodiment, the electrode comprises a porous material.

In an embodiment, the ablation device also has a fluid conduit coupled to the porous material of the electrode configured to deliver fluid to the porous material of the electrode.

In an embodiment, the ablation device also has a pressure sensor, coupled to a component of the ablation device, which generates a signal indicative of pressure in at least one of the fluid chamber and the low pressure chamber.

In an embodiment, the pressure sensor generates a signal indicative of pressure in the low pressure chamber.

In an embodiment, the ablation device also has a controller operatively coupled to the ablative element and the pressure sensor, the controller operating the ablative element based, at least in part, on the signal indicative of pressure.

In an embodiment, the controller ceases operation of the ablative element if the signal indicative of pressure is less than a minimum pressure value.

In an embodiment, the controller begins operation of the ablative element based, at least in part, on the signal indicative of pressure is greater than a minimum pressure value.

In an embodiment, the pressure sensor is positioned in the low pressure chamber.

In an embodiment, the pressure sensor generates a signal indicative of attachment of the ablation device to the tissue.

In an embodiment, the ablation device also has a controller operatively coupled to the ablative element and the pressure sensor, the controller operating the ablative element based, at least in part, on the signal indicative of attachment.

In an embodiment, the ablation device also has a flow sensor, operably coupled to a component of the ablation device, which generates a signal indicative of a flow of the fluid in at least one of the fluid chamber and the low pressure chamber.

In an embodiment, the flow sensor generates a signal indicative of fluid flow in the low pressure chamber.

In an embodiment, the ablation device also has a controller operatively coupled to the ablative element and the flow sensor, the controller operating the ablative element based, at least in part, on the signal indicative of fluid flow.

In an embodiment, the controller ceases operation of the ablative element if the signal indicative of fluid flow is less than a minimum flow value.

In an embodiment, the controller begins operation of the ablative element based, at least in part, on the signal indicative of fluid flow is greater than a minimum flow value.

In an embodiment, the flow sensor is positioned in the fluid chamber.

In an embodiment, the source of low pressure is fluidly coupled to the low pressure chamber with a suction port.

In an embodiment, the ablation device also has an anti-occlusion structure to prevent occlusion of the suction port by the tissue.

In an embodiment, the anti-occlusion structure comprises a mesh screen positioned between the suction port and the tissue.

In an embodiment, the anti-occlusion structure comprises a post positioned proximate the suction port approximately parallel with the inner wall.

In an embodiment, the anti-occlusion structure comprises a plurality of posts positioned within the low pressure chamber approximately parallel with the inner wall.

In an embodiment, a method of ablating tissue with the ablation device has the steps of placing the open face of the fluid chamber and the low pressure chamber against the tissue. Then a low pressure is created in the low pressure chamber, thereby at least partially securing the ablation device to the tissue and at least partially fluidly isolating the fluid chamber from the low pressure chamber. Then the tissue is ablated with the ablation element.

In an embodiment, the creating low pressure step completely fluidly isolates the fluid chamber from the low pressure chamber.

In an embodiment, the method also has the step of delivering a source of fluid to the fluid chamber.

In an embodiment, the step of delivering a source of fluid to the fluid chamber comprises delivering the fluid through a reservoir fluidly coupled to the fluid chamber.

In an embodiment, the step of delivering a source of fluid step delivers a conductive fluid.

In an embodiment, the ablative element comprises a porous material, and the delivering a source of fluid step delivers the conductive fluid through the porous material.

In an embodiment, the method also has the step of removing the fluid from the fluid chamber.

In an embodiment, the creating low pressure step creates the low pressure having a pressure lower than a pressure in the fluid chamber.

In an embodiment, the outer wall comprises a flange which contacts the tissue, and further comprising the step of conforming the flange to conform to the tissue.

In an embodiment, the ablation device further comprises a pressure sensor, coupled to the ablation device, which generates a signal indicative of pressure, and the ablating step occurs based, at least in part, on the signal indicative of pressure.

In an embodiment, the ablation device further comprises a controller operatively coupled to the pressure sensor, and the controller executes the ablation step based, at least in part, on the signal indicative of pressure.

In an embodiment, the ablation device also has a flow sensor, coupled to the ablation device, which generates a signal indicative of fluid flow, and the ablating step occurs based, at least in part, on the signal indicative of pressure.

In an embodiment, the ablation device also has a controller operatively coupled to the pressure sensor, and the controller executes the ablation step based, at least in part, on the signal indicative of fluid flow.

### DRAWINGS

The accompanying drawings are included to provide a further understanding of embodiments and are incorporated in and constitute a part of this specification. The drawings illustrate embodiments and together with the description serve to explain principles of embodiments. Other embodiments and many of the intended advantages of embodiments will be readily appreciated as they become better understood by reference to the following detailed description. The elements of the drawings are not necessarily to scale relative to each other. Like reference numerals designate corresponding similar parts.

FIG. 1 is a schematic diagram of an environment of the present disclosure;

FIG. 2 is a schematic diagram of an example of an ablation device of the present disclosure;

FIG. 3 is a side-sectional schematic diagram of the ablation device of FIG. 2;

FIGS. 4A is an isometric view of an example ablation device constructed in accordance with the example shown in FIG. 2;

FIG. 4B is another isometric view of the example ablation device of FIG. 4A;

FIG. 5 is a schematic side-sectioned view of the example ablation device shown in FIG. 4A and taken along lines 5A-5A;

FIG. 5B illustrates a cross-section of the length of device in FIG. 4A along lines 5B-5B.

FIGS. 6A-6D are side views of various embodiments of an outer wall of the ablation device of FIG. 5; and

FIGS. 7A and 7B are cross-sectional views of an embodiment of the ablation device of FIG. 5, incorporating structures which may help prevent patient tissue from blocking suction ports, and FIG. 7C is a bottom view of the embodiment of FIGS. 7A and 7B.

### DESCRIPTION

The entire contents of U.S. Provisional Patent Application No. 61/046,531, Batchelor et al, Suction Force Ablation Device, filed Apr. 21, 2008, is incorporated herein by reference.

In the following description, reference is made to the accompanying drawings, which form a part hereof, and in which is shown by way of illustration specific embodiments in which the invention may be practiced. In this regard, directional terminology, such as “top,” “bottom,” “front,” “back,” “leading,” “trailing,” etc., is used with reference to the orientation of the Figure(s) being described. Because components of embodiments can be positioned in a number of different orientations, the directional terminology is used for purposes of illustration and is in no way limiting. It is to be understood that other embodiments may be utilized and structural or logical changes may be made without departing from the scope of the present disclosure. The following detailed description, therefore, is not to be taken in a limiting sense. It is also to be understood that the features of the various exemplary embodiments described herein may be combined with each other, unless specifically noted otherwise.

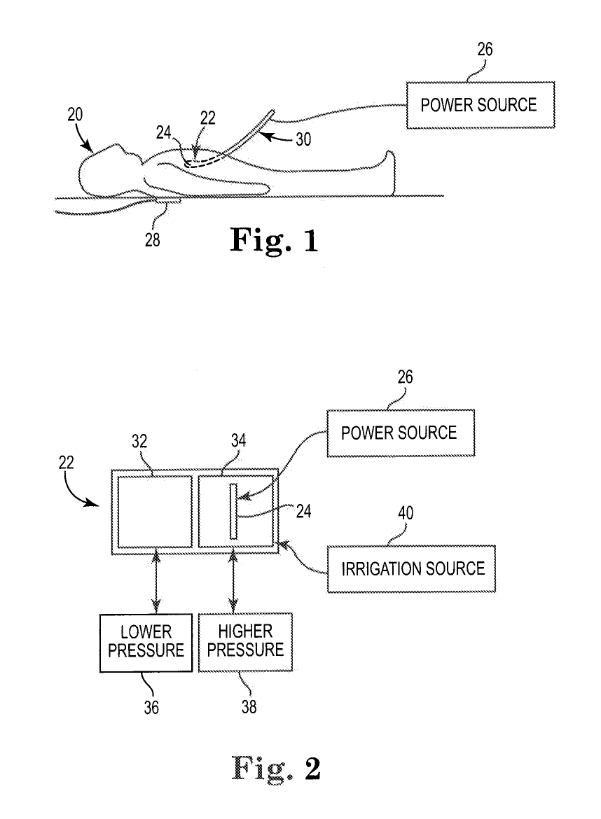

FIG. 1 illustrates an example environment of this disclosure. FIG. 1 illustrates a patient 20 having an ablation device 22 disposed within a body cavity of the patient during surgery. Often, the tissue to be ablated within the patient includes tissue on a beating heart, such as the endocardial or epicardial tissue of the heart. Other body organ tissue, such as the liver, lungs or kidney, may also be ablated. Tissue types that may be ablated include skin, muscle or even cancerous tissue or abnormal tissue growth.

The ablation device 22 includes at least one conductive element 24 such as an ablation electrode. In various embodiments, multiple conductive elements 24 may be provided. In such embodiments, conductive elements 24 may include elements such as pacing electrodes and sending electrodes, and other functions not involving the delivery of ablation energy. In an embodiment, the conductive element 24 is electrically coupled to a power source 26. The embodiment includes conductive element 24 at the distal end of the device. The illustrated embodiment also includes an indifferent or non-ablating electrode 28 that serves as a return plate for energy transmitted through conductive element 24 in cases where the ablation device 22 is unipolar. The non-ablating electrode 28 can be placed elsewhere on the patient's body other than the ablation site. For example, electrode 28 can be placed on the patient's back, thigh or shoulder.

The ablation device 22 can be any suitable ablation tool such as, for example, a catheter, an electrocautery device, an electrosurgical device, a suction-assisted ablation tool, an ablation pod, an ablation paddle, an ablation hemostat, an ablation wire, or the like. The device 22 and its components can be made of a biocompatible material such as stainless steel, biocompatible epoxy or biocompatible plastic. In general, a biocompatible material prompts little allergenic response from the patient's body and is resistant to corrosion from being placed within the patient's body. Also, the biocompatible material causes no or minimal additional stress to the patient's body. The biocompatibility of a material can be created or enhanced by coating the material with a biocompatible coating.

The ablation device 22 can be permanently or removably attached to a maneuvering apparatus for manipulating the device onto a tissue surface, such as a handle and shaft 30. In various embodiments, shaft 30 may be articulated or otherwise bendable. In an embodiment, ablation device 22 may be positioned on a jaw of a hemostat or related device. In alternative embodiments, an ablation device 22 may be positioned on each of the jaws of a hemostat or related device. The ablation device 22 may also be maneuvered with a leash or pull-wire assembly or positioned on a pen-like maneuvering apparatus such as the Sprinkler pen, marketed as the Cardioblate pen, available from Medtronic, Inc. Also, any appropriate flexible, malleable or rigid handle, or any appropriate endoscopic or thoroscopic-maneuvering apparatus could be used as a maneuvering apparatus.

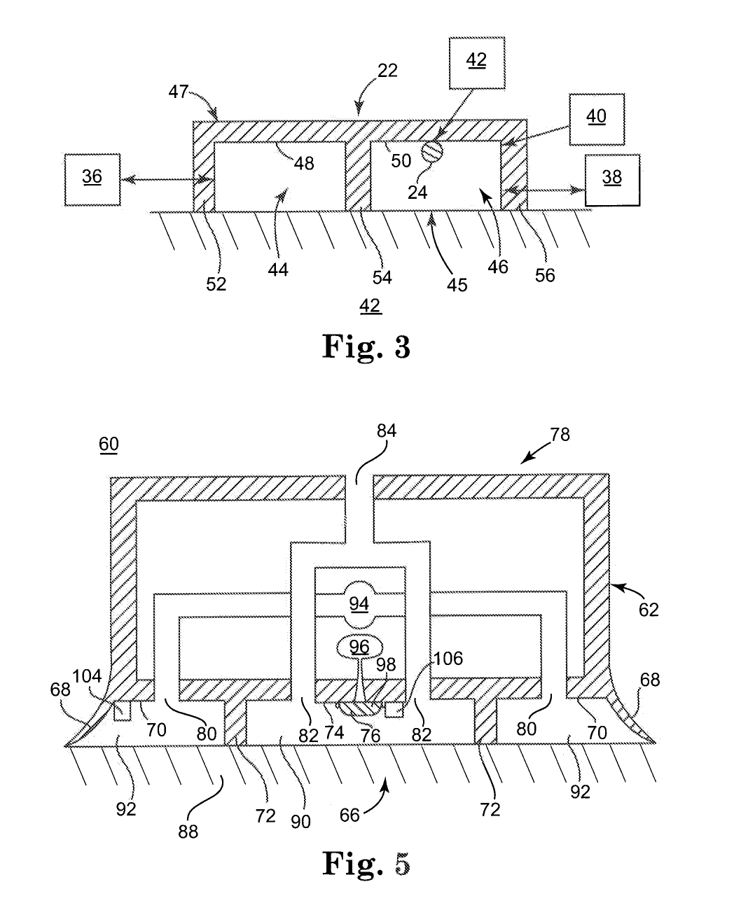

FIG. 2 illustrates an example ablation device 22. The device includes a first portion 32 and a second portion 34. When the device is urged against organic tissue, the first portion and second portion form first and second chambers that are fluidically isolated from each other in that the two chambers are not in fluid communication with each other. Each chamber is isobaric. The first portion 32 is in fluid communication with a low pressure 36, such as a suction port or vacuum device. The second portion is in fluid communication with a pressure 38 that is a higher pressure than the low pressure 38. In one example, the pressure 38 is atmospheric pressure or generally 760 mmHg (101.325 kPa), and the low pressure is a suction source at a relative pressure of 300 mmHg (40.0 kPa). The ablation electrode 24 is located within the second portion 34, and is electrically coupled to the power source 26. Further, the second portion 34 can be coupled to an irrigation source 40 that can provide an irrigation fluid around the electrode 24.

In operation, the ablation device is held in place against the tissue with the suction from the first chamber 44. The tissue is ablated with the electrode 24 in the second chamber 46. The issue of proper balance of pressure is addressed in that the second chamber 46 is set at 1 atmosphere and does not vary due to irrigation fluid 40 or other pressure introduced into the chamber 46. The fluidically isolated chambers 44, 46 prevent the irrigation fluid 40 from entering into the first chamber 44 and prevent the low pressure source from affecting the second chamber 46. The device 22 provides for good adherence to the tissue as well as optimal ablation effects.

Generally, adherence to tissue is a significant element in a successful ablation procedure. Vacuum applied to first chamber 44 assists with holding ablation device 22 in contact with patient tissue. In an embodiment, the amount of vacuum may be modulated, or otherwise controlled, in order to control the amount of suction available in first chamber 44 to assist with holding ablation device 22 in contact with patient tissue. The higher the vacuum (lower pressure with respect to ambient) in first chamber 44, the greater force with which ablation device 22 may be held against patient tissue. In an embodiment, the amount of vacuum available in first chamber 44 may be adjusted or adjustable either automatically by sensing a degree or force of contact, such as with a pressure switch or sensor or contact switch or sensor, or may be manually adjusted, such as by an operator.

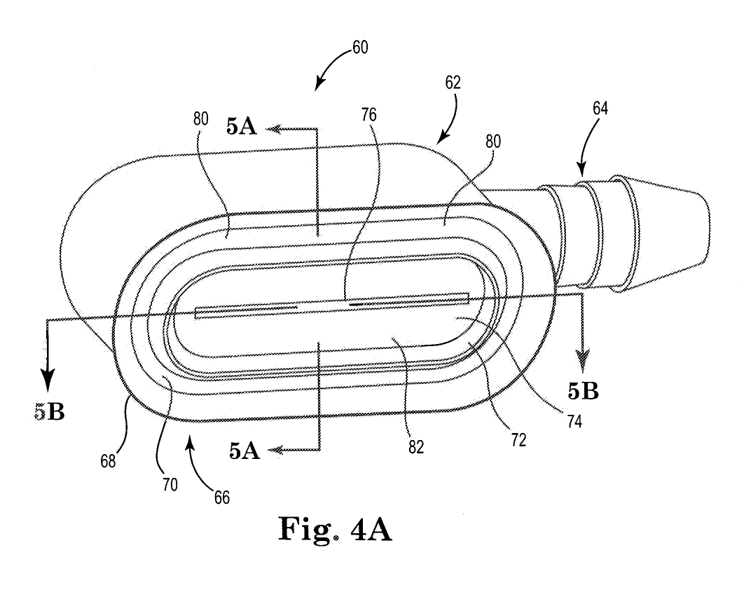

FIG. 3 illustrates an example of the device 22 having a face 45 and a back 47, where the face 45 is urged against the organic tissue 42 to form a first chamber 44 and a second chamber 46. The first chamber 44 includes a first recessed surface 48 and the second chamber 46 includes a second recessed surface 50. Walls 52 and 54 are also included in forming the first chamber 44. In the example shown, walls 54 and 56 are also included in forming the second chamber 46. Wall 54 is a common wall to both chambers 44, 46, and in the example extends a length from the recessed surfaces 48, 50 and is of a proper shape to maintain fluid isolation between the chambers. Wall 52 is of a proper length and shape so as to maintain a low-pressure area in the first chamber 44. The electrode 24 is included within the second chamber 46. In the example shown the electrode 24 is connected to the second recessed surface 50, although other options are possible and can include adding an additional support structure within the second chamber 46 and attaching the electrode to the addition support structure.

In one example, the face 45 of the device 22 is adapted to conform to the surface of the tissue when positioned against the target tissue. The device 22 or selected portions of the device can be made from a flexible material, such as a pliable polymer, biocompatible rubber thermoplastic elastomer, or PVC. In another example, the device 22 or portions of the device can be made of a more rigid material covered with an elastic material over the face 45. The low pressure source 36 applied through device 22 can cause device or face 45 to conform more closely to the shape of the target tissue. The device 22 may also be made of a malleable stainless steel or other material that is shapeable but not necessary flexible or of a conductive polymer. In one example, one or more walls can be made from flexible closed cell foam.

The ablation device 22 may be colored so that it can be easily visible against the target tissue. Alternatively, the device 22 may be clear to provide less distraction to the surgeon or to provide viewing of any material perhaps being suctioned.

The electrode 24 of the ablation device 22 can be permanently or removably connected to the power source 26. This energy is typically electrical, such as radiofrequency (RF) energy. In some examples, however, it may also be any appropriate type of energy such as, for example, microwave or ultrasound energy.

The electrode 24 can be constructed of stainless steel, platinum, other alloys, or a conductive polymer. In examples where the device 22 is includes of a more flexible material, the electrodes 24 can be made of materials that would flex with the device. Such flexible electrodes may be, for example, made in a coil or spring configuration. Flexible electrodes 24 can also be made from a gel, such as a hydrogel. Also, the electrode 24 can in some cases deliver fluid, such as, for example, a microporous electrode, a “weeping” electrode, or an electrode made of a hydrogel.

The ablation device 22 can also be permanently or removably attached to at least one irrigation source 40 for irrigating the ablation site with a fluid. In some examples, the ablation site may not be irrigated. Fluid is conveyed to the site via fluid openings within the second chamber 46, which in one example are integrated into the electrode 24. In other examples the fluid may be delivered to the site via a separate irrigation mechanism, such as an irrigation pump. Also, fluid openings may be disposed in any appropriate manner in the second chamber 46.

The irrigation fluid may be any suitable fluid such as saline, an ionic fluid that is conductive or another conductive fluid. The irrigating fluid can serve to cool the electrode 24 of ablation device 22. Irrigated ablation is also known to create deeper lesions that are more likely to be transmural. The application of fluid to an ablation site may also prevent electrodes, particularly metal electrodes, from contacting the target tissue. Direct contact of electrodes to the target tissue may char or burn the tissue, which may clog the device. Furthermore, continuous fluid flow may keep the ablation device surface temperature below the threshold for blood coagulation, which can also clog the device. Use of irrigating fluid can therefore reduce the need to remove a clogged ablation device for cleaning or replacement.

Ionic irrigation fluid also serves to conduct energy. The presence of an ionic fluid layer between electrode 24 and the tissue to be ablated may also ensure that an ionic fluid layer conforming to the tissue contours is created. In one example, saline solution is used. Alternatively, other energy-conducting liquids, such as Ringer's solution, ionic contrast, or even blood, may be used. Diagnostic or therapeutic agents, such as Lidocaine, CA.sup.++ blockers, or gene therapy agents may also be delivered before, with, or after the delivery of the irrigating fluid.

Although not shown, the device 22 can include at least one temperature-sensitive element. These elements may be, for example, thermocouple wires, thermisters or thermochromatic inks. These elements allow temperature to be measured to provide information as to whether adjustments to the device or the procedure should be made. For example, too high a temperature can char the tissue or cause the blood at the ablation site to coagulate, and too little temperature can cause ineffective ablation. Preferably, the elements contact the tissue proximate the ablation. The tissue is allowed to heat until the thermocouple elements indicate a temperature that usually indicates cell death (such as, for example, 15 seconds at 55 degrees Celsius), which can also indicate that the lesion is transmural. Thermocouple elements that may be used include 30 gauge type T thermocouple wire from Dodge Phelps Company. Also, a type of conductive epoxy which may be used to fasten the elements to the device 22 is epoxy number BA-2902, which is available from Trecon.

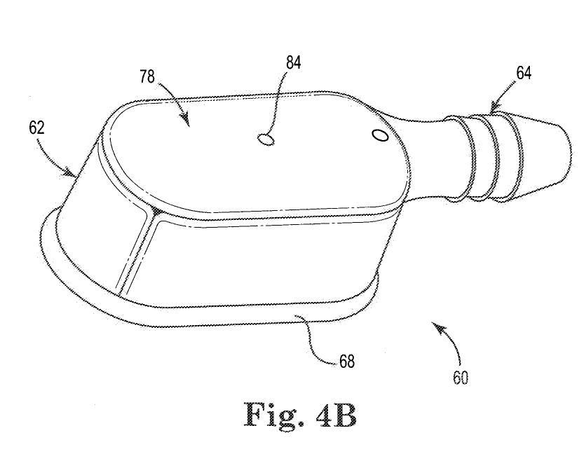

FIGS. 4A and 4B illustrate a particular example of an ablation device 60 constructed in accordance with this disclosure. The device 60 includes a housing 62, a connector 64, and a face portion 66. The face portion 66 includes an outer wall 68, a first recessed surface 70, an inner wall 72, and a second recessed surface 74. The second recessed surface 74 includes an electrode 76 such as a fluid-eluting polymer coupled to the irrigation source. Alternatively, electrode 76 may be made, at least in part, from a porous material fluidly coupled to irrigation source 40. The porous material may help distribute fluid evenly throughout fluid chamber 46. In further alternative embodiments, electrode may be made of sintered metal or other material with relatively small holes to permit the permeation of fluid. FIG. 4B also illustrates the back 78 of the device 60.

The face portion 66 includes suction ports 80 and vents 82. The first recessed surface 70 includes a plurality of suction ports 80. The specific placement and number of suction ports 80 may vary. The suction ports 80 are in fluid communication with a suction conduit that extends out of the connector 64 and connects with a suction source such as a port in an operating room. The second recess surface 74 includes a plurality of vents 82 that are in fluid communication to atmospheric pressure, such as through at least one rear vent 84 on the housing 62, or specifically on the back 78 as indicated in FIG. 4B. The vents 84 also serve to channel away excess irrigation fluid.

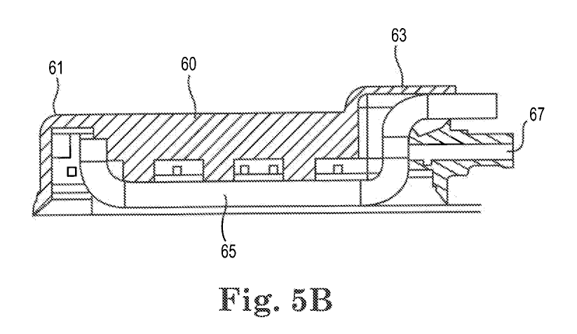

FIG. 5A schematically illustrates a cross-section of the width of device 60 in FIGS. 4A and 4B taken along lines 5a-5a. FIG. 5 shows the face 66 urged against organic tissue 88. Two chambers 90, 92 are created with the device 60 against tissue 88. The inner wall 72 serves to create an inner chamber 90 at atmospheric pressure. The inner wall 72 and outer wall 68 serve to create an outer chamber 92 that surrounds the inner chamber 90. The outer chamber 92 is under a low pressure so a suction force holds the device 60 against the tissue 88. The inner wall 72 also serves to fluidically isolate the two chambers 90, 92, so that the inner chamber 90 remains at atmospheric pressure and the outer chamber 92 remains at a lower pressure.

The vents 82 are shown in communication with the rear vent 84. The suction ports 80 are shown in communication with each other and a suction conduit 94. The fluid-eluting electrode 76 is shown in communication with an irrigation conduit 96, and the electrode also includes a power coupling 98. The suction conduit 94, irrigation conduit 96, and power coupling 98 extend outside of the device 60, and preferably out through the connector 64 to the respective low pressure source, irrigation source, and power source.

Pressure sensor 104 may be included in ablation device 60 within chamber 92. Pressure sensor 104 may provide feedback on the efficacy of the suction coupling between ablation device 60 and tissue 42. Based on the response from pressure sensor 104, ablation energy may be delivered if the pressure is such that ablation device 60 is likely secured against tissue 42. Alternatively, ablation energy delivery may be disabled if the response from pressure sensor 104 is such that ablation device 60 is likely not secured against tissue 42.

In an embodiment, pressure sensor 104 may be based around a Pressurex™ thin film pressure sensor, which provides pressure distribution and magnitude between two contacting or impacting surfaces. Alternative thin film pressure sensors may be utilized. In alternative embodiments, other forms of pressure sensors may be utilized to measure pressure within chamber 92, within chamber 90, or within both chambers 90 and 92. Depending on the nature of pressure sensor 104, the enable/disable based on measured pressure function may be automatic when pressure sensor 104 is coupled to a controller which is operable to control the delivery of ablation energy automatically. Alternatively, the enable/disable function may be user-operated, depending on the capabilities of the particular pressure sensor 104 utilized.

Alternatively, pressure sensor 104 may be positioned on or in other components of ablation device 60, including, but not limited to, on outer wall 68, on inner wall 72, and within fluid chamber 90. Multiple pressure sensors 104 may be positioned in the various locations. Pressure sensors 104 may utilize a variety of different shapes, including rings around the edges of outer wall 68 or inner wall 72, elongate shapes, or may be relatively more discrete sensors. In such a configuration, the one or more pressure sensors 104 may provide an indication that pressure sensor 104, and, by extension, ablation device 60 in general, is in contact with patient tissue 42 or other surfaces. On the basis of the signal of tissue contact, a user may initiate the delivery of ablation energy. Alternatively, a controller may, based at least in part on the indication of contact with a surface, automatically being delivery of ablation energy.

FIG. 5B illustrates a cross-section of the length of device 60 in FIGS. 4A and 4B along lines 5B-5B. Distal end 61 is the end of device 60 away from user control, fluid and vacuum sources. Proximal end 63 is the end of device 60 proximate user control, fluid and vacuum sources. In an embodiment, conduit 65 is coupled to irrigation source 40 and conduit 67 is coupled to low pressure source 36. In such an embodiment, fluid may flow generally from distal end 61 to proximal end 63. Alternatively, fluid may flow from proximal end 63 to distal end 61. In an alternative embodiment, conduit 65 is coupled to low pressure source 36 and conduit 67 is coupled to irrigation source 40.

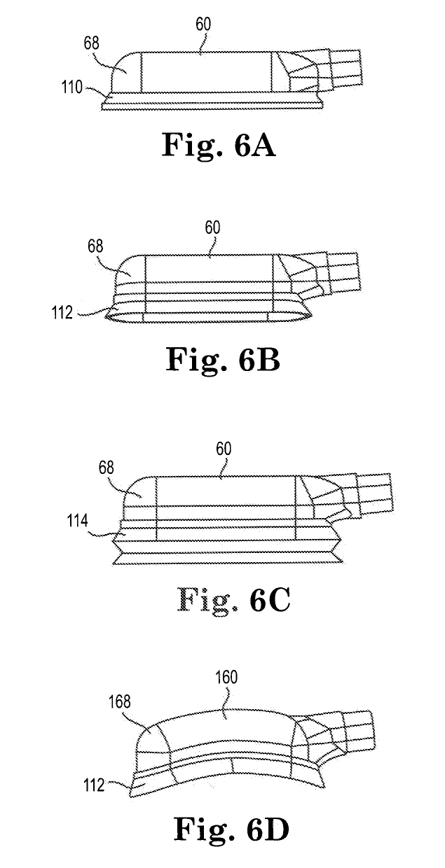

As illustrated, outer wall 68 is flanged to help secure an isobaric outer chamber 92, although other configurations of each wall are envisioned. In such configurations, outer wall 68 may be rigid or flexible to help secure an isobaric outer chamber 92. In an embodiment, outer wall 68 is flexible. The structure of outer wall 68 may be varied in alternative embodiments, illustrated in FIGS. 6A-6D. As illustrated in FIG. 6A, outer wall 68 of ablation device 60 may incorporate gasket 110 to facilitate the creation of a fluid seal between outer wall 68 and tissue 42. As shown in FIG. 6B, outer wall 68 of ablation device 60 may alternatively be a single bellows 112, which may be consistent with the embodiment of FIG. 5. Alternatively, as shown in FIG. 6C, double bellows 114 may be utilized, which may, in various embodiments, improve an ability to create isobaric chamber 92. In a further embodiment, illustrated in FIG. 6D, outer wall 168 of ablation device 160 may be curved for use on patient tissue 42 that is itself curved. Alternatively, curved ablation device 160 may be utilized on non-curved patient tissue, particularly if outer wall 168 is flexible. As illustrated, outer wall 168 has a single bellows, but alternative configurations are envisioned. Such configurations may include embodiments consistent with gasket 110 and double bellows 114.

Flow sensor 106 may be positioned in chamber 90 to measure the flow of saline or other fluid into chamber 90. In an embodiment, when the fluid flow falls below a minimum level a warning may be provided to a user. The warning may be an audible alarm, a visual notification, a tactile warning, or any alternative warning or alarm suitable to warn a user. In an alternative embodiment, depending on the particular flow sensor 106 utilized, the delivery of ablation energy may be automatically halted if the flow of fluid into chamber 90 falls below a particular level. In a further embodiment, a warning may be provided if the fluid flow falls below a first level and an automatic cutoff may be provided if the fluid flow falls below a second level.

Suction ports 80 may become blocked or occluded by tissue 42, which may reduce or eliminate the suction force from suction port 80 and increase the pressure in the corresponding chamber. In various embodiments, structures may be positioned to reduce or prevent tissue 42 from occluding or otherwise blocking suction ports 80.

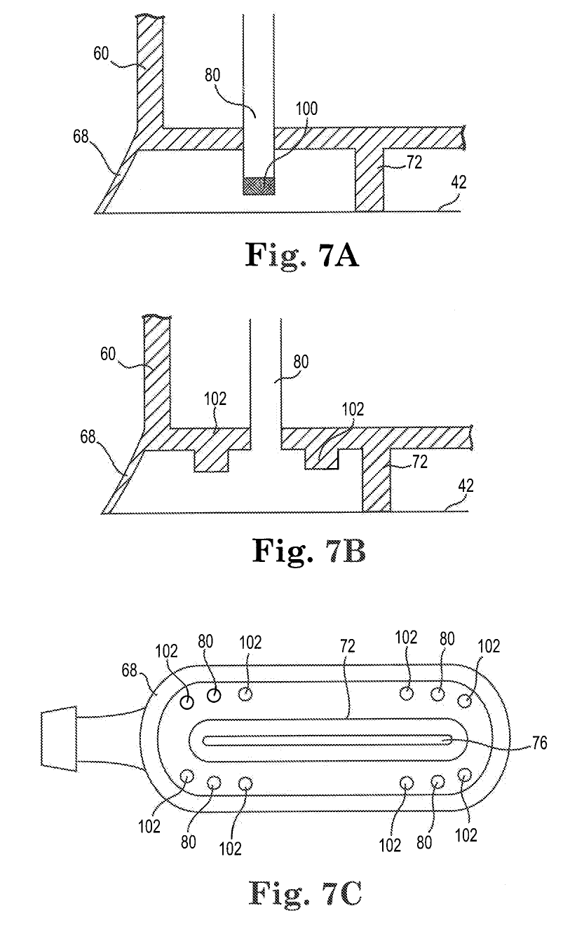

In an embodiment illustrated in FIG. 7A, screen 100, such as a mesh screen, may be positioned around suction ports 80. A distance between screen 100 and suction port 80 may be selected such that the distance is sufficient to prevent suction from being reduced or cut off altogether by tissue 42. Variations in distance may be dependent on the characteristics of tissue 42, on the suction force, and other factors which may be present on a case-by-case basis. Alternative screens to mesh screen 100 may be utilized, in various embodiments providing permeability to but restraining patient tissue 42 against the suction force from suction port 80. In such embodiments, mesh screen 100 or an alternative screen may be detachable from ablation device 60 and replaceable with an alternative embodiment of mesh screen 100 or alternative screen.

In an alternative embodiment, illustrated in FIGS. 7B and 7C, posts 102 may be arrayed to obstruct the progression of tissue 42 toward suction port 80. In the illustrated embodiment, posts 102 are arrayed throughout ablation device 60. In an alternative embodiment, two posts 102 are arrayed directly around suction port 80. In alternative embodiments, one post 102 may be utilized, or three or more posts 102 may be positioned around suction port 80. Height and width of post 102 may be selected dependent on the number of posts 102 utilized, the characteristics of tissue 42, on the suction force, and other factors which may be present on a case-by-case basis.

Interaction and interference between screen 100 and tissue 42 and between posts 102 and tissue 42 may provide additional adherence between ablation device 60 and tissue 42. Such additional adherence may serve to compliment suction from suction ports 80. Characteristics of screen 100 and posts 102 may be selected to enhance the interference in order to increase an amount of grip between screen 100/posts 102 and tissue 42.

Various alternative structures to obstruct tissue 42 from contacting or nearing suction port 80 are envisioned. Such structures include, but are not limited to bars positioned laterally with respect to suction port 80, in contrast to the horizontal orientation of posts 102, and permeable membranes.

Although specific embodiments have been illustrated and described herein, it will be appreciated by those of ordinary skill in the art that a variety of alternate and/or equivalent implementations may be substituted for the specific embodiments shown and described without departing from the scope of the present invention. This application is intended to cover any adaptations or variations of the specific embodiments discussed herein. Therefore, it is intended that this invention be limited only by the claims and the equivalents thereof.

What is claimed is:

1. A suction force ablation device suitable for use with an organic tissue, said device comprising:
a first recessed area configured to form a first chamber when said device is urged against said tissue;
a second recessed surface configured to form a second chamber, concentric with said first chamber, when said device is urged against said tissue, wherein said first and second chambers are fluidically isolated from each other; and
an electrode disposed within said second chamber
wherein said first chamber is under a low pressure than said second chamber said first chamber and is configured to provide a suction force against said tissue.

2. The device of claim 1 wherein said first recessed area and said second recessed areas are each surrounded by a wall.

3. The device of claim 2 wherein said wall is configured to be flexible and conform to said tissue.

4. The device of claim 3 wherein said wall is constructed from a flexible closed cell foam.

5. The device of claim 2 wherein a common wall separated said first recessed area and said second recessed area.

6. The device of claim 5 wherein said first recessed area surrounds said second recessed area.

7. The device of claim 1 wherein said electrode is electrically coupled to a power source.

8. The device of claim 1 wherein an irrigation fluid is introduced into said first chamber from an irrigation source.

9. The device of claim 8 wherein said electrode is a fluid eluting polymer fluidly coupled to said irrigation source.

10. A suction force ablation device suitable for use with an organic tissue, said device comprising:
a housing having first upstanding wall and a second upstanding wall, said first upstanding wall being concentric with said second upstanding wall;
a first recessed portion surrounded by said first wall;
an electrode disposed within said first wall;
a vent within said first wall, wherein said vent is in fluid communication with atmospheric pressure through another vent on said housing;
a second recessed portion surrounded by said first wall and said second wall; and
a suction port disposed between said first and second walls, wherein said suction port is configured to provide a suction force.

11. The device of claim 10 wherein said second wall surrounds said second recessed layer and said first wall.

12. The device of claim 10 wherein said device is configured to be urged against tissue and form a first chamber surrounded by said first wall and said second wall and a second chamber surrounded by said first walls, wherein said first chamber surrounds said entire second chamber.

13. The device of claim 10 wherein said housing further includes a connector portion.

14. An ablation device for ablating tissue, comprising:
an outer wall and an inner wall, approximately parallel and concentric with said outer wall, defining an inner fluid chamber and an outer low pressure chamber, each of said outer wall and said inner wall having an edge defining an open face of said fluid chamber and said low pressure chamber;
an ablative element contained within said fluid chamber; and
a source of low pressure coupled to said low pressure chamber;
wherein, when said edge of said outer wall and said edge of said inner wall contact a surface, said ablation device is at least partially secured to said surface by low pressure created in said low pressure chamber by said source of low pressure;
said fluid chamber being at least partially fluidly isolated from said low pressure chamber when said ablation device is at least partially secured to said surface.

15. The ablation device of claim 14 further comprising a source of fluid delivered to said fluid chamber.

16. The ablation device of claim 15 wherein said source of fluid comprises a reservoir fluidly coupled to said fluid chamber.

17. The ablation device of claim 15 wherein the fluid is a conductive fluid.

18. The ablation device of claim 15 further comprising a fluid removal lumen fluidly coupled to said fluid chamber.

19. The ablation device of claim 14 wherein a fluid chamber pressure is greater than a low pressure chamber pressure.

20. The ablation device as in claim 14 wherein said outer wall comprises a flange which contacts said surface.

21. The ablation device as in claim 20 wherein said flange is flexible.

22. The ablation device as in claim 20 wherein said flange is curved.

23. The ablation device as in claim 14 wherein said outer wall comprises a bellows.

24. The ablation device as in claim 23 wherein said bellows is a single bellows.

25. The ablation device as in claim 23 wherein said bellows is a double bellows.

26. The ablation device as in claim 14 wherein said low pressure chamber is defined by a gap between said outer wall and said inner wall and said fluid chamber is defined by said inner wall.

27. The ablation device as in claim 26 wherein said low pressure chamber forms a concentric ring around said fluid chamber.

28. The ablation device as in claim 15 wherein said ablative element comprises an electrode.

29. The ablation device as in claim 28 wherein said electrode comprises a porous material.

30. The ablation device as in claim 29 further comprising a fluid conduit coupled to said porous material of said electrode configured to deliver fluid to said porous material of said electrode.

31. The ablation device as in claim 14 further comprising a pressure sensor, coupled to a component of said ablation device, which generates a signal indicative of pressure in at least one of said fluid chamber and said low pressure chamber.

32. The ablation device as in claim 31 wherein said pressure sensor generates a signal indicative of pressure in said low pressure chamber.

33. The ablation device as in claim 32 further comprising a controller operatively coupled to said ablative element and said pressure sensor, said controller operating said ablative element based, at least in part, on said signal indicative of pressure.

34. The ablation device as in claim 33 wherein said controller ceases operation of said ablative element if said signal indicative of pressure is less than a minimum pressure value.

35. The ablation device as in claim 33 wherein said controller begins operation of said ablative element based, at least in part, on said signal indicative of pressure is greater than a minimum pressure value.

36. The ablation device as in claim 31 wherein said pressure sensor is positioned in said low pressure chamber.

37. The ablation device as in claim 31 wherein said pressure sensor generates a signal indicative of attachment of said ablation device to said tissue.

38. The ablation device as in claim 34 further comprising a controller operatively coupled to said ablative element and said pressure sensor, said controller operating said ablative element based, at least in part, on said signal indicative of attachment.

39. The ablation device as in claim 15 further comprising a flow sensor, operably coupled to a component of said ablation device, which generates a signal indicative of a flow of said fluid in at least one of said fluid chamber and said low pressure chamber.

40. The ablation device as in claim 39 wherein said flow sensor generates a signal indicative of fluid flow in said low pressure chamber.

41. The ablation device as in claim 40 further comprising a controller operatively coupled to said ablative element and said flow sensor, said controller operating said ablative element based, at least in part, on said signal indicative of fluid flow.

42. The ablation device as in claim 41 wherein said controller ceases operation of said ablative element if said signal indicative of fluid flow is less than a minimum flow value.

43. The ablation device as in claim 41 wherein said controller begins operation of said ablative element based, at least in part, on said signal indicative of fluid flow is greater than a minimum flow value.

44. The ablation device as in claim 39 wherein said flow sensor is positioned in said fluid chamber.

45. The ablation device as in claim 14 wherein said source of low pressure is fluidly coupled to said low pressure chamber with a suction port.

46. The ablation device as in claim 45 further comprising an anti-occlusion structure to prevent occlusion of said suction port by said tissue.

47. The ablation device as in claim 46 wherein said anti-occlusion structure comprises a mesh screen positioned between said suction port and said tissue.

48. The ablation device as in claim 46 wherein said anti-occlusion structure comprises a post positioned proximate said suction port approximately parallel with said inner wall.

49. The ablation device as in claim 46 wherein said anti-occlusion structure comprises a plurality of posts positioned within said low pressure chamber approximately parallel with said inner wall.

50. A method of ablating tissue with an ablation device as in claim 14, comprising the steps of:
placing said open face of said fluid chamber and said low pressure chamber against said tissue; then
creating low pressure in said low pressure chamber, thereby at least partially securing said ablation device to said tissue and at least partially fluidly isolating said fluid chamber from said low pressure chamber; and then
ablating said tissue with said ablation element.

51. The method of claim 50 wherein said creating low pressure step completely fluidly isolates said fluid chamber from said low pressure chamber.

52. The method of claim 50 further comprising the step of delivering a source of fluid to said fluid chamber.

53. The method of claim 52 wherein the step of delivering a source of fluid to said fluid chamber comprises delivering said fluid through a reservoir fluidly coupled to said fluid chamber.

54. The method of claim 52 wherein the step of delivering a source of fluid step delivers a conductive fluid.

55. The method of claim 54 wherein said ablative element comprises a porous material, and wherein said delivering a source of fluid step delivers said conductive fluid through said porous material.

56. The method of claim 52 further comprising the step of removing said fluid from said fluid chamber.

57. The method of claim 50 wherein said creating low pressure step creates said low pressure having a pressure lower than a pressure in said fluid chamber.

58. The method of claim 50 wherein said outer wall comprises a flange which contacts said tissue, and further comprising the step of conforming said flange to conform to said tissue.

59. The method of claim 50 wherein said ablation device further comprises a pressure sensor, coupled to said ablation device, which generates a signal indicative of pressure, and wherein said ablating step occurs based, at least in part, on said signal indicative of pressure.

60. The method of claim 59 wherein said ablation device further comprises a controller operatively coupled to said pressure sensor, and wherein said controller executes said ablation step based, at least in part, on said signal indicative of pressure.

61. The method of claim 50 wherein said ablation device further comprises a flow sensor, coupled to said ablation device, which generates a signal indicative of fluid flow, and wherein said ablating step occurs based, at least in part, on said signal indicative of pressure.

62. The method of claim 61 wherein said ablation device further comprises a controller operatively coupled to said pressure sensor, and wherein said controller executes said ablation step based, at least in part, on said signal indicative of fluid flow.
